(19) United States
(12) Patent Application Publication
(10) Pub. No.: US 2008/0095865 A1
(43) Pub. Date: Apr. 24, 2008

(54) COMPOSITION AND METHOD FOR INCREASING LEAN MUSCLE MASS, DECREASING MUSCLE LOSS, INCREASING MUSCLE STRENGTH AND IMPROVING ATHLETIC PERFORMANCE

(75) Inventors: Marvin HEUER, Mississauga (CA); Shan CHAUDHURI, Mississauga (CA); Ken CLEMENT, Mississauga (CA)

Correspondence Address: TORYS LLP, 79 WELLINGTON ST. WEST, SUITE 3000, TORONTO, ON, M5K 1N2, CA

(73) Assignee: APLODAN FORMULATIONS LTD., Mississauga (CA)

(21) Appl. No.: 11/925,156

(22) Filed: Oct. 26, 2007

Related U.S. Application Data
(63) Continuation-in-part of application No. 11/240,267, filed on Sep. 29, 2005.
(63) Continuation-in-part of application No. 11/656,713, filed on Jan. 22, 2007.
Application No. 11/656,713 is a division of application No. 11/240,267, filed on Sep. 29, 2005.
(63) Continuation-in-part of application No. 11/534,861, filed on Sep. 25, 2006, now abandoned.
(60) Provisional application No. 60/614,134, filed on Sep. 29, 2004.
(60) Provisional application No. 60/614,134, filed on Sep. 29, 2004.

(51) Int. Cl. A61K 31/385; A61K 36/258

(52) U.S. Cl. 424/728; 514/440

(57) ABSTRACT

A dietary supplement comprising Creatinol O-Phosphate and Alpha Lipoic Acid, wherein the dietary supplement optionally comprises a timed-release or controlled-release delivery system. The timed-release or controlled-release delivery system may include one of quick release, slow release and controlled release delivery system. By way of oral administration, the present invention provides a method increasing lean muscle mass, increasing muscle strength and improving athletic performance.

### RELATED APPLICATIONS

This application is a continuation-in-part of U.S. patent application Ser. No. 11/240,267 which is related to and claims the benefit of priority to U.S. Provisional Patent Application Ser. No. 60/614,134, entitled “Nutritional Composition for Promoting Lean Muscle Mass and Enhancing Muscle Performance and Endurance,” filed on Sep. 29, 2004, and a continuation-in-part of U.S. patent application Ser. No. 11/656,713, which is a divisional application of U.S. patent application Ser. No. 11/240,267, and a continuation-in-part of U.S. patent application Ser. No. 11/534,861, filed on Sep. 25, 2006, the disclosures of which are hereby fully incorporated by reference herein.

### FIELD OF THE INVENTION

The present invention relates to a nutritional composition for promoting muscle performance and acting as a hydrogen (H+) blocker, increasing lean muscle mass, improving athletic performance, and a method of providing same by consuming the nutritional composition and a method of manufacturing such a nutritional composition.

### SUMMARY OF THE INVENTION

The foregoing needs and other needs and objectives that will become apparent for the following description are achieved in the present invention which comprises a nutritional composition and method for promoting muscle performance and acting as a hydrogen (H+) blocker, increasing lean muscle mass, increasing muscle strength and improving athletic performance. In a preferred embodiment, the nutritional composition can be used as a dietary supplement.

The present invention provides a composition and method comprising at least a combination of Creatinol-O-Phosphate and Alpha Lipoic Acid and is directed towards increasing muscle strength, increasing lean muscle mass, and improving athletic performance.

The present invention also provides, in some embodiments, for a timed release delivery system for a nutritional composition. For the purposes of this invention, “time-released” should be understood as including any type of timed release delivery system, including but not limited to any type of quick-release delivery system, slow release delivery system and/or controlled release delivery system, such as a multi-phasic temporal release profile.

### BACKGROUND OF THE INVENTION

Creatine use has been thoroughly studied and it is well-establish as a beneficial dietary supplement for replenishing energy stores in working muscle cells (Greenhaff P L, Bodin K, Soderlund K, Hultman E. Effect of oral creatine supplementation on skeletal muscle phosphocreatine resynthesis. Am J. Physiol. 1994 May; 266(5 Pt 1):E725-30), thereby increasing strength, reducing fatigue resulting from high-intensity exercise (Greenhaff P L, Casey A, Short A H, Harris R, Soderlund K, Hultman E. Influence of oral creatine supplementation of muscle torque during repeated bouts of maximal voluntary exercise in man. Clin Sci (Lond). 1993 May; 84(5):565-71) as well as increasing muscle growth (Volek J S, Duncan N D, Mazzetti S A, Staron R S, Putukian M, Gomez A L, Pearson D R, Fink W J, Kraemer W J. Performance and muscle fiber adaptations to creatine supplementation and heavy resistance training. Med Sci Sports Exerc. 1999 August; 31(8):1147-56).

Insulin is involved in the carbohydrate-induced uptake of glucose by cells. The release of glucose from cells is also concomitantly inhibited by insulin and its storage as glycogen and triglycerides is promoted (Khan A H, Pessin J E. Insulin regulation of glucose uptake: a complex interplay of intracellular signalling pathways. Diabetologia. 2002 November; 45(11):1475-83). Additionally, insulin has also been shown to stimulate the uptake of amino acids by muscle cells and stimulate protein synthesis (Biolo G, Declan Fleming R Y, Wolfe R R. Physiologic hyperinsulinemia stimulates protein synthesis and enhances transport of selected amino acids in human skeletal muscle. J Clin Invest. 1995 February; 95(2):811-9), particularly following exercise (Biolo G, Williams B D, Fleming R Y, Wolfe R R. Insulin action on muscle protein kinetics and amino acid transport during recovery after resistance exercise. Diabetes. 1999 May; 48(5):949-57) which is required for increasing muscle mass and associated muscle function, i.e. strength.

Alpha Lipoic Acid has been shown to have insulin-like activity in terms of glucose metabolism (Streeper R S, Henriksen E J, Jacob S, Hokama J Y, Fogt D L, Tritschler H J. Differential effects of lipoic acid stereoisomers on glucose metabolism in insulin-resistant skeletal muscle. Am J Physiol. 1997 July; 273(1 Pt 1):E185-91; Ziegler D, Hanefeld M, Ruhnau K J, Hasche H, Lobisch M, Schutte K, Kerum G, Malessa R. Treatment of symptomatic diabetic polyneuropathy with the antioxidant alpha-lipoic acid: a 7-month multicenter randomized controlled trial (ALADIN III Study). ALADIN III Study Group. Alpha-Lipoic Acid in Diabetic Neuropathy. Diabetes Care. 1999 August; 22(8):1296-301). Likely due to this insulin-like activity, the combination of Alpha Lipoic Acid and Creatine, via supplementation has been shown to result in markedly improved uptake and retention of Creatine by skeletal muscle cells (Burke D G, Chilibeck P D, Parise G, Tamopolsky M A, Candow D G. Effect of alpha-lipoic acid combined with creatine monohydrate on human skeletal muscle creatine and phosphagen concentration. Int J Sport Nutr Exerc Metab. 2003 September; 13(3):294-302).

### DETAILED DESCRIPTION OF THE INVENTION

In the following description, for the purposes of explanation, numerous specific details are set forth in order to provide a thorough understanding of the present invention. It will be apparent, however, to one of ordinary skill in the art that the present invention may be practised without these specific details.

Creatinol O-Phosphate

Creatinol O-Phosphate, also known by the synonyms, (N-methyl-N-(beta-hydroxyethyl) guanidine O-phosphate, creatinol-phosphate, COP, and (CAS: 6903-79-3), is a phosphoric ester derivative of creatinol. The chemical structure of creatinol O-phosphate is as follows:

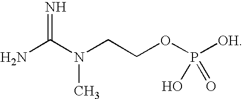

As a supplement, Creatinol O-Phosphate has been shown to be well tolerated and without side effects (Melloni G F, Minoja G M, Lureti G F, Merlo L, Pamparana F, Brusoni B. Acute clinical tolerance of creatinol O-phosphate. Arzneimittelforschung. 1979, 29(9a):1447-9). Early studies of Creatinol-O-Phosphate explored its use as a treatment for heart lesions and also its ability to restore reduced cardiac contractile function. As a result of these early studies, Creatinol-O-Phosphate has been successfully used to improve cardiac parameters in patients with inadequate coronary blood flow (Barlattani M, Guglielmi G, Mammarella A. Creatinol O-phosphate therapy in patients with inadequate coronary circulation. Double-blind clinical trial. Arzneimittelforschung. 1979; 29(9a): 1483-5). Additionally, early investigators of the properties of Creatinol-O-Phosphate hypothesized that the cardioprotective effect of Creatinol-O-Phosphate was due to action on anaerobic glycolysis (Godfraind T, Saleh M M. Action of creatinol-O-phosphate on the contractility changes evoked by hypoxia and ischemia in rat isolated heart. Arzneimittelforschung. 1984; 34(9):968-72). Furthermore, an additional or alternative possible mechanism hypothesized in regard to Creatinol-O-Phosphate is an effect in the electrophysiological properties of the cell membrane as shown by other investigators (Botti G, Bonatti V. Preliminary report on electrophysiological effectiveness of creatinol O-phosphate (COP) in human subjects. Arzneimittelforschung. 1979; 29(9a):1491-4).

Creatinol-O-Phosphate, in addition to its cardio-protective effects, has been has also been shown to improve muscle development and increase the capacity of a muscle to perform physical activity. In one study, hand-grip strength was improved by Creatinol-O-Phosphate administration, while remaining unaffected in a placebo group (Nicaise J. Creatinol O-phosphate (COP) and muscular performance: a controlled clinical trial. Curr Ther Res Clin Exp. 1975, 17(6):531-4). Moreover, in another study conducted in elderly subjects, it was found that Creatinol-O-Phosphate improved muscular performance as compared to controls (Cavalieri U, Quadri A, Ghirardi F. Effects of creatinol o-phosphate on the muscle function of elderly people. The Therapeutic Clinic. 1974, 69: 215-223).

Creatinol-O-Phosphate functions in a similar manner to that of Creatine, which is well known is in art, in addition to possibly possessing unique properties. Creatinol-O-Phosphate administration has been found to increase urine levels of creatinine (Melloni G F, Minoja G M, Lureti G F, Merlo L, Pamparana F, Brusoni B. Acute clinical tolerance of creatinol O-phosphate. Arzneimittelforschung. 1979, 29(9a): 1447-9), the end metabolite and degradation product of creatine and phosphocreatine, which diffuses out of cells for later excretion by the kidneys. This metabolism to Creatinol-O-Phosphate may be due to phosphodiesterases contained within the body. Creatinol-O-Phosphate may therefore enhance athletic performance and muscle growth in a manner similar to that of creatine, aiding in the rapid rephosphorylation of ATP to provide energy to a given muscle (Greenhaff P L, Bodin K, Soderlund K, Hultman E. Effect of oral creatine supplementation on skeletal muscle phosphocreatine resynthesis. Am J. Physiol. 1994, 266(5 Pt 1):E725-30).

Although the present invention is not to be limited by any theoretical explanation, it is believed that Creatinol-O-Phosphate may be involved in and enhances ATP synthesis, which is reduced during muscular exercise. Creatinol-O-Phosphate has been shown to increase muscular strength in human subjects, which is also marked in the elderly, as well as reduce asthenia and muscular weakness in convalescent subjects. It is believed that Creatinol-O-Phosphate allows for more endurance in the form of muscular contraction before fatigue sets in.

As used herein, a serving of the nutritional composition comprises from about 0.1 g to about 10 g of creatinol-O-phosphate. More preferably, a serving of the nutritional composition comprises from about 0.5 g to about 5 g of Creatinol-O-Phosphate, and most preferably about 1.5 grams, twice daily.

In various embodiments of the present invention which are set forth in greater detail below in the examples below, a dietary supplement includes Creatinol-O-Phosphate or derivatives thereof. A serving of the dietary supplement may include from about 0.250 g to about 5.0 g of Creatinol-O-Phosphate or derivatives thereof. The preferred dosage, in a serving of said dietary supplement, comprises about 1.0 g of Creatinol-O-Phosphate or derivatives thereof.

Alpha Lipoic Acid

As used herein, “Alpha Lipoic Acid” (ALA) preferably refers to the chemical compound 1,2-dithiolane-3-pentanoic acid, CAS registry No. 62-46-4, also known as, thioctic acid and 6,8-dithio octanoic acid. As used herein, “Alpha Lipoic Acid” also includes derivatives of Alpha Lipoic Acid such as esters, and amides, as well as other derivatives, including derivatives that become active upon metabolism. The chemical structure of Alpha Lipoic Acid is as follows:

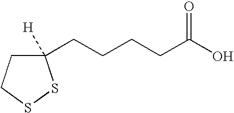

Alpha Lipoic Acid is a co-enzyme found in the cellular energy-producing structures, the mitochondria. Moreover, ALA works in synergy with vitamins C and E as an antioxidant in both water- and fat-soluble environments.

Negative age-related changes in mitochondrial function, accumulated oxidative damage and metabolic rate were all improved (Hagen T M, Ingersoll R T, Lykkesfeldt J, Liu J, Wehr C M, Vinarsky V, Bartholomew J C, Ames A B. (R)-alpha-lipoic acid-supplemented old rats have improved mitochondrial function, decreased oxidative damage, and increased metabolic rate. FASEB J. 1999 February; 13(2):411-8) in rats supplemented with ALA, thereby indicating its protective effects in these parameters. As such, the antioxidant activity of ALA is likely involved in the prevention of cell death due to an improvement in oxidative stress (Arivazhagan P, Juliet P, Panneerselvam C. Effect of dl-alpha-lipoic acid on the status of lipid peroxidation and antioxidants in aged rats. Pharmacol Res. 2000 March; 41(3):299-303). Furthermore, ALA has been linked to a beneficial increase in high-density lipoproteins (Wollin S D, Wang Y, Kubow S, Jones P J. Effects of a medium chain triglyceride oil mixture and alpha-lipoic acid diet on body composition, antioxidant status, and plasma lipid levels in the Golden Syrian hamster. J Nutr Biochem. 2004 July; 15(7):402-10) possibly due to its known effects as an antioxidant.

Alpha Lipoic Acid is an insulin modulator and an antioxidant that serves as protection against oxidative injury in non-neuronal and neuronal tissue. Alpha Lipoic Acid is a nutrient that the human body makes in minute quantities and may also be obtained from yeast and liver. Studies have shown that Alpha Lipoic Acid can significantly increase the body's utilization of blood sugar in type II diabetics and that lipoic acid may increase the metabolic clearance rate of glucose by 50% in diabetics. In Europe, Alpha Lipoic Acid has been used as a substitute for insulin in the treatment of Type II diabetes.

Although the present invention is not to be limited by any theoretical explanation, it is believed that insulin is a primary factor that stimulates glucose and creatine transport into the muscle cells and that Alpha Lipoic Acid both mimics and enhances the actions of insulin in glucose and creatine transport into the muscle cells.

Further to its insulin-like activity, Alpha Lipoic Acid may enhance the uptake and retention of supplemental Creatinol-O-Phosphate or derivatives thereof in a manner similar to that of the enhanced Creatine retention induced by Alpha Lipoic Acid with respect muscle cells (Burke D G, Chilibeck P D, Parise G, Tamopolsky M A, Candow D G. Effect of alpha-lipoic acid combined with creatine monohydrate on human skeletal muscle creatine and phosphagen concentration. Int J Sport Nutr Exerc Metab. 2003 September; 13(3):294-302) thereby enhancing the effects of Creatinol-O-Phosphate or derivatives thereof through an increase in retention.

Preferably, a serving of the nutritional composition comprises from about 0.1 mg to about 10 mg of Alpha Lipoic Acid per gram of nutritional composition. More preferably, a serving of the nutritional composition comprises from about 0.5 mg to about 5 mg of Alpha Lipoic Acid per gram of nutritional composition.

In another embodiment of the present invention, which is set forth in greater detail in the examples below, a dietary supplement may include Alpha Lipoic Acid or derivatives thereof. A serving of the dietary supplement may include from about 0.005 g to about 0.1 g of Alpha Lipoic Acid or derivatives thereof. The preferred dosage of a serving of the dietary supplement comprises about 0.010 g of Alpha Lipoic Acid or derivatives thereof.

In additional embodiments, the dietary supplement may further comprise N-Acetyl-Cysteine, Taurine, Chromium, Vanadium and Ginseng in combinations or as single ingredient additions to the Creatinol-O-Phosphate and Alpha Lipoic Acid.

N-Acetyl-Cysteine

N-acetyl cysteine (NAC) is the N-acetyl derivative of the amino acid L-cysteine. N-acetyl cysteine is a precursor of glutathione, which is a potent antioxidant. Glutathione can not cross the cell membrane, but N-acetyl cysteine easily crosses the cell membrane where it is converted to cysteine and, subsequently, glutathione. Reactive oxygen species (ROS) such as hydrogen peroxide and hydroxyl-free radicals reduce intracellular and extracellular concentrations of glutathione. N-acetyl cysteine is an efficient way to replenish glutathione and reduce damage caused by ROS.

L-taurine

L-taurine is an amino acid that is not involved in the synthesis of proteins in animals and is the end product of L-cysteine metabolism. L-taurine is the principle free intracellular amino acid found in human tissue. L-taurine is also an antioxidant, and has been shown to improve insulin sensitivity.

Chromium

Chromium has been shown to improve insulin sensitivity and glucose disposal. Chromium is supplied as a chromium chelate. Preferred chromium chelate includes chromium picolinate and chromium nicotinate. Most preferred chromium chelate is chromium nicotinate or polynicotinate.

Vanadium (Vanadyl Sulfate)

Vanadium is a trace mineral. Vanadium appears to be important in normal bone growth and as a cofactor for various enzyme reactions. Vanadium inhibits ATPases, phosphatases, and phosphoryl-transfer enzymes. Some evidence suggests that vanadium can mimic the actions of insulin, possibly by causing phosphorylation of insulin receptor proteins. Vanadium activates the receptor; stimulates glucose oxidation and transport; inhibits lipolysis in adipose tissue; stimulates glycogen synthesis in the liver; inhibits hepatic gluconeogenesis; inhibits intestinal glucose transport; and increases glucose uptake, utilization, and glycogen synthesis in skeletal muscle.

Ginseng

As used herein, Ginseng preferably refers to American Ginseng (Panax quinquefolium L.), or Korean Ginseng (Panax Ginseng) or Siberian Ginseng ((Eleutherococcus senticosus) and derivatives thereof. Most preferably Ginseng refers to American Ginseng (Panax quinquefolium L.), or Korean Ginseng (Panax Ginseng) and derivatives thereof.

While not being bound by theory it is believed that Ginseng stimulates the production of nitric oxide.

Timed Release Delivery System

The present invention also provides for a timed release delivery system for a nutritional composition for providing any of the above-mentioned effects, which comprises a combination of pharmaceutical grade excipients comprising Hydroxypropyl Methyl Cellulose (Methocellulose K100M Premium CR EP-Hypromellose 2208 USP XXII), Ethyl Cellulose (Ethocellulose Standard FP Premium—NF XVII), and Hydroxypropylcellulose (Klucel LF—NF XVII).

The present invention also provides for a timed release delivery system for providing any of the above-mentioned effects, which comprises a hydrophilic caplet matrix system comprising water-soluble (i.e., Hydroxypropyl Methyl Cellulose and Hydroxypropylcellulose) and water-insoluble (i.e., Ethyl Cellulose) polymers uniformly incorporated at a specified ratio.

In a timed release delivery system for a nutritional composition for providing any of the above-mentioned effects, the hydrophilic caplet matrix system is in the ratio of 1 Hydroxypropylcellulose:1 Ethyl Cellulose:0.5 Hydroxypropyl Methyl Cellulose.

According to an embodiment of the invention, the timed release feature may provide a hydrocolloid-coated gel-forming fiber. This caplet containing the dietary ingredients provides a unique, efficient and controllable prolonged-action drug-delivery system. The solid prolonged (timed/extended) release technology consists of an effective dose of particles of a biologically-absorbable dietary ingredients, gel-forming dietary matrix particles, a mineral salt of carbonate or bicarbonate which releases a physiologically-acceptable gas upon ingestion, and a physiologically-acceptable acid, the combination of the fiber and mineral salt providing amounts which effect prolonged but effective release of the dietary ingredients upon oral ingestion and exposure of the caplets to biological fluids.

The gel-forming dietary fiber may comprise about 35% to about 50% by weight, the mineral salt may comprise about 5% to about 75% by weight, the physiologically-acceptable acid may comprise 0% to about 50% by weight, and the ratio of the weight of the gel-forming fiber to the weight of the drug or therapeutic agent may be between about 1,000:0.5 and 1:1.5, the gel-forming dietary fiber particles being coated with a hydrocolloid film and the dietary ingredients being optionally coated with a film of the same or a different hydrocolloid.

By precoating the gel-forming fiber or gum particles, and optionally but advantageously also the dietary ingredients, with a film of Sodium Carboxymethylcellulose (NACMC) or other hydrocolloid, including hydrocolloids such as, e.g., natural and modified gums, celluloses and modified celluloses, pectin, mucillages, modified starches, noncellulosic polysaccharides, algal polysaccharides, and mixtures thereof, particularly preferred hydrocolloids including carboxymethyl cellulose, methyl cellulose, karaya gum, acacia gum, sodium alginate, calcium alginate, hydroxypropylmethylcellulose, and mixtures thereof, and then tableting or granulating together with a mineral carbonate or bicarbonate to speed up hydration, the locking up or sealing from further hydration can be prevented and a smooth and controlled release of the dietary supplement can be achieved.

Compositions that include such an extended release delivery system may prolong the release of dietary ingredients upon administration to a user. Such a composition may work up to 10 to 12 hours after ingestion i.e., the dietary ingredients being released and staying in the blood stream for several hours.

Furthermore, additional embodiments of the present invention may be incorporated into a specific controlled-release solid dosage form. U.S. Non-Provisional patent application Ser. No. 11/709,525 entitled “Method for a Supplement Dietary composition having a Multi-Phasic Dissolution Profile, filed Feb. 21, 2007, which is herein fully incorporated by reference, discloses a method of achieving a solid oral dosage form with multiple dissolution characteristics for the release of active ingredient, thereby providing a controlled-release format. Conventional oral dosage formulations are bound by the rate of bioavailability of the substance upon oral administration. This particularly problematic for poorly-soluble compounds which have an inherently low rate of dissolution, such as Alpha Lipoic Acid.

It is herein understood that, due to the relationship between solubility and dissolution, the amount of a substance in solution at any given time is dependent upon both dissolution and solubility. Furthermore, it is understood by way of extension that increasing the rate of dissolution of a given substance acts to reduce the time to dissolution of a given solute or substance in a given solvent. However, the absolute solubility of said solute does not increase with infinite time. Thus, increasing the rate of dissolution of a substance with increase the amount of said substance in solution at earlier point in time, thus increase the rate of bioavailability of said substance at earlier time upon oral administration.

According the method disclosed in U.S. Non-Provisional patent application Ser. No. 11/709,525, a controlled-release format can be created by varying the size of the particles of a given substance in the solid oral dosage form. The size of the particles is controlled by a micronization process which can produce fine powders from solid compounds. The powders, with varying particle sizes can then be formed into a solid oral dosage form. In order to achieve a controlled-release profile, such as, for example a multi-phasic release profile, the ratio of particle sizes relative each other is varied to produce the desired profile.

The present invention is directed to a nutritional composition comprising at least Creatinol-O-Phosphate and Alpha Lipoic Acid.

According to this invention, the nutritional composition may be consumed in any form. For instance, the dosage form of the nutritional composition may be provided as, e.g., a capsule, a tablet, a caplet, a liquid beverage, a powder beverage mix (including forms that provide an effervescent effect and those without such an effervescent effect), a dietary gel, or as a ready-to-eat bar or drink product.

Furthermore, the dosage form of the nutritional composition in accordance with this embodiment may be provided in accordance with customary processing techniques for herbal and/or dietary supplements in any of the forms mentioned above. For instance, in the embodiment in which the nutritional composition is provided as a capsule, the active ingredients may be suitably processed and encapsulated into capsules, e.g., cellulose, gelatin, etc., with suitable excipients. Those of skill in the art will appreciate that the nutritional composition may contain a variety of excipients. Still further, the nutritional composition in accordance with this embodiment may be provided in a timed release, e.g., quick-release, slow release and controlled release, delivery system. Additional details of various possible time release delivery systems are provided below.

In an embodiment of the present invention, the dietary supplement or other nutritional composition comprises at least a combination of Creatinol-O-Phosphate or derivatives thereof and Alpha Lipoic Acid or derivatives thereof. While not wishing to be bound by theory, it is understood by the inventors that the beneficial activity of supplemental Creatinol-O-Phosphate, is improved through the combination of at least Creatinol-O-Phosphate and Alpha Lipoic Acid in a single dietary supplement wherein an increase in the uptake and retention of Creatinol-O-Phosphate results from the insulin-like activity of Alpha Lipoic Acid.

The present invention, according to various embodiments thereof, is directed methods for increasing muscle strength, increasing lean muscle mass, and improving athletic performance. An embodiment of the present invention comprises the administration of a composition comprising at least Creatinol-O-Phosphate or derivatives thereof and Alpha Lipoic Acid or derivatives thereof.

It is understood by the inventors that advantageously, the combination of Alpha Lipoic Acid and Creatinol-O-Phosphate with subsequent co-administration in the form of a dietary supplement will enhance the retention of Creatinol-O-Phosphate in the body.

According to various embodiments of the present invention, the dietary supplement may be consumed in any form. For instance, the dosage form of the diet supplement may be provided as, e.g., a powder beverage mix, a liquid beverage, a ready-to-eat bar or drink product, a capsule, a liquid capsule, a tablet, a caplet, or as a dietary gel. The preferred dosage form of the present invention is as a caplet or tablet.

Furthermore, the dosage form of said dietary supplement may be provided in accordance with customary processing techniques for dietary supplements in any of the forms mentioned above. Additionally, the dietary supplement set forth in the example embodiments herein may contain any appropriate number and type of excipients, as is well-known in the art.

The present dietary supplement or those similarly envisioned by one of ordinary skill in the art, may be utilized in compositions and methods for increasing lean muscle mass, increasing muscle strength and improving athletic performance.

Preferably, the dietary supplement of the present invention is consumed by an individual in accordance with the following method: As a dietary supplement, a serving of said dietary supplement may be administered by means of consumption in conjunction with a liquid medium at least one (1) time daily wherein each serving is comprised of at least one (1) caplet or tablet. Said dietary supplement may be consumed immediately post-workout, pre-workout, or in the morning upon waking on non-workout days. In this manner, the dietary supplement may increase lean muscle mass, increase muscle strength and improve athletic performance.

Although the following example illustrates the practice of the present invention in one of its embodiments, the example should not construed as limiting the scope of the invention. Other embodiments will be apparent to one of ordinary skill in the art from consideration of the specifications and example.

### EXAMPLES

### Example 1

A serving of the nutritional composition comprises the following ingredients

| Ingredient #: | Ingredients |
|---|---|
| 1 | Creatinol |
| 2 | Alpha Lipoic Acid |

### Example 2

A serving of the nutritional composition comprises the following ingredients

| Ingredient #: | Ingredients |
|---|---|
| 1 | Creatinol |
| 2 | Alpha Lipoic Acid |
| 3 | NAC |
| 4 | Taurine |
| 5 | Chromium |
| 6 | Vanadium |
| 7 | Ginseng |

### Example 3

The ingredients of the dietary supplement, supplied in caplet form, may be consumed with 8 ounces of water. The composition of the dietary supplement includes Creatinol-O-Phosphate (1.0 g) and Alpha Lipoic Acid (0.010 g). The dietary supplement may be consumed at least one (1) time daily wherein said serving is consumed immediately following exercise.

### Example 4

The ingredients of the dietary supplement, supplied in tablet form, may be consumed with 8 ounces of water. The composition of the dietary supplement includes Creatinol-O-Phosphate (1.0 g) and Alpha Lipoic Acid (0.010 g). The dietary supplement may be consumed at least one (1) time daily wherein said serving is consumed immediately following exercise.

### Example 5

The ingredients of the dietary supplement, supplied in caplet form, may be consumed with 8 ounces of water. The composition of the dietary supplement includes Creatinol-O-Phosphate (1.0 g) and Alpha Lipoic Acid (0.010 g). The dietary supplement may be at least one (1) time daily wherein said serving is consumed prior to exercise.

### Example 6

The ingredients of the dietary supplement, supplied in tablet form, may be consumed with 8 ounces of water. The composition of the dietary supplement includes Creatinol-O-Phosphate (1.0 g) and Alpha Lipoic Acid (0.010 g). The dietary supplement may be at least one (1) time daily wherein said serving is consumed prior to exercise.

### Example 7

The ingredients of the dietary supplement, supplied in tablet form, may be consumed with 8 ounces of water. The composition of the dietary supplement includes Creatinol-O-Phosphate (1 g) and Alpha Lipoic Acid (0.010 g). The dietary supplement may be consumed at least one (1) time daily wherein said serving is consumed upon waking in the morning.

### Example 8

The ingredients of the dietary supplement, supplied in caplet form, may be consumed with 8 ounces of water. The composition of the dietary supplement includes Creatinol-O-Phosphate (1.0 g) and Alpha Lipoic Acid (0.010 g). The dietary supplement may be consumed at least one (1) time daily wherein said serving is consumed upon waking in the morning.

Extensions and Alternatives

In the foregoing specification, the invention has been described with specific embodiments thereof however; it will be evident that various modifications and changes may be made thereto without departing from the broader spirit and scope of the invention.

What is claimed:

1. A dietary supplement comprising at least Creatinol-O-Phosphate and Alpha Lipoic Acid.

2. The dietary supplement of claim 1 wherein:
about 1.0 g of Creatinol-O-Phosphate is provided;
about 0.010 g of Alpha Lipoic Acid is provided.

3. A method of increasing lean muscle mass in an individual comprising at least the step of administering the composition of claim 2 to the individual.

4. A method of increasing muscle strength in an individual comprising at least the step of administering the composition of claim 2 to the individual.

5. A method of improving athletic performance in an individual comprising at least the step of administering the composition of claim 2 to the individual.

6. The diet supplement of claim 1, further comprising creatine, NAC, taurine, chromium, vanadium, and ginseng.

7. The diet supplement of claim 1, wherein the dietary supplement includes a timed release delivery system and wherein the timed release delivery system is one of quick release, slow release and controlled release.
